(19) United States
(12) Patent Application Publication
(10) Pub. No.: US 2023/0340131 A1
(43) Pub. Date: Oct. 26, 2023

(54) STABLE AQUEOUS HIGH CONCENTRATION FORMULATION OF INTEGRIN ANTIBODY

(71) Applicant: DR. REDDY’S LABORATORIES LIMITED, Hyderabad (CN)

(72) Inventors: Murali JAYARAMAN, Kancheepuram (Dist) (IN); Swapnil Vasudeo PAKHALE, Jalgaon Dist (IN); Shrija Ghosh GHOSH, Lucknow (IN)

(21) Appl. No.: 18/036,546

(22) PCT Filed: Nov. 12, 2021
(86) PCT No.: PCT/IN2021/051065

§ 371 Date: May 11, 2023

(30) Foreign Application Priority Data
Nov. 12, 2020 (IN) 202041049481

(51) Int. Cl. C07K 16/28; A61K 47/18; A61K 47/02

(52) U.S. Cl. CPC C07K 16/2839; A61K 47/183; A61K 47/02; A61K 39/39591

(57) ABSTRACT

The present invention discloses a high concentration formulation of an α4β7antibody, comprising α4β7antibody, amino acid(s), salt and surfactant, wherein the free amino acid(s) are hydrophobic amino acids and/or basic amino acid (s). The disclosed antibody formulations are liquid formulations that are also suitable for different mode of administration (subcutaneous/intravenous) and exhibits stability under various accelerated conditions.

### FIELD OF THE INVENTION

The present invention is related to a stable aqueous high concentration formulations of an antibody molecule, wherein the antibody formulation is stabilized with minimal excipients. The disclosed formulations stabilizes the antibody from about 50 mg/ml to about 200 mg/ml which are suitable for intravenous or subcutaneous route of administration.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody. In particular, high concentration liquid formulations are susceptible to degradation and/or aggregation. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

Hence, designing a formulation that is stable at high or low concentrations of the therapeutic protein/antibody, aiding in different route of administration (intravenous or subcutaneous), pose a significant developmental challenge. Further, every protein or antibody with its unique characteristics and properties of degradation, adds to the complexity in the development of a stable formulation and may demand a specific formulation.

A stable formulation of a therapeutic protein or antibody involves addition of a wide variety of buffers, stabilizers/excipients including amino acids, sugars, polyols, surfactants, salts, polymers, amines, anti-oxidants, chelators etc. Many of the FDA approved therapeutic proteins /antibodies contain more than one category of stabilizers.

A formulation combination with increased concentration of protein and/or stabilizers may increase the viscosity of the formulation, in turn increasing the injection time and pain at the site of injection and also pose difficulties during processing of the drug substance. Hence, it is necessary to develop an improved formulation, which contains minimal number or concentration of excipients, yet stabilizing the drug at a wide range of its concentration.

### SUMMARY

The present invention discloses a high concentration formulation of an α4β37 antibody comprising, the said antibody, histidine-phosphate buffer, free amino acid(s), sodium chloride and surfactant, wherein the free amino acid(s) are hydrophobic amino acids and/or basic amino acid (s). The concentration of the antibody stabilized by the present invention/formulation is greater than 50 mg/ml, in particular the formulation stabilizes the antibody at concentrations from about 150 mg/ml to 180 mg/ml.

The antibody in the said formulation is stable and maintains at least 97% of monomeric content of the antibody in the formulation even after storage for four weeks at 40° C. Further, the disclosed high concentration α4β7 antibody formulations of the invention exhibit stability at room temperature (at 25° C.) for at least four weeks and maintains at least 98% of monomeric content.

The disclosed formulations of the invention are devoid of sugar/sugar alcohol, polyethyleneglycol (PEG) and anti-oxidant.

The invention also discloses a process of controlling charge variants and aggregation/fragmentation of α4β7 antibody in an α4β7 antibody formulation by formulating the antibody in a buffer composition comprising arginine, sodium chloride and surfactant. In particular, the change in high molecular weight species (i.e. aggregates) and low molecular weight species (i.e. fragments) of the antibody for high concentration vedolizumab (at least 150 mg/ml) formulation is less than 1.5%, more specifically less than 1% even when the antibody samples are subjected to accelerated condition of 40° C. for four weeks or at 25° C. for four weeks.

The invention also discloses a method to impart colloidal stability to α4β7 antibody by formulating the antibody in a buffer comprising arginine and sodium chloride.

### DETAILED DESCRIPTION OF THE INVENTION

### Definitions

The term “about” refers to a range of values that are similar to the stated reference value and includes a range of values that fall within 20% or less of the stated reference value.

The term “antibody” refers to a glycoprotein comprising at least two heavy (H) chains and two light (L) chains inter-connected by disulfide bonds, or an antigen-binding portion thereof. The “antibody” as used herein encompasses whole antibodies or any antigen binding fragment (i.e., “antigen-binding portion”) or fusion protein thereof.

The term “stable” formulation refers to the formulation wherein the antibody therein retains its physical stability and/or chemical stability and/or biological activity upon storage.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Positively charged molecules bind to anion exchange resins while negatively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof.

The term sugar/s as used herein includes sugars and sugar alcohols/polyols. Sugars can be referred to monosaccharides, disaccharides, and polysaccharides. Examples of sugars include, but are not limited to, sucrose, trehalose, glucose, dextrose, raffinose and others. Examples of sugar alcohols or polyols include, but are not limited to, mannitol, sorbitol, and others. The disclosed formulations of the invention are devoid of sugar/sugar alcohol.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

The term “free amino acid” as used herein refers to amino acid that is included in the formulation and is not a part of the buffer component. An amino acid may be present in its D- and/or L-form. The amino acid may be present as any suitable salt e.g. a hydrochloride salt, such as Arginine-HCI.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “antioxidant” refers to an agent that inhibits the oxidation of other molecules and is not part of buffer component. Examples of antioxidants herein include citrate, methionine, lipoic acid, uric acid, glutathione, tocopherol, carotene, lycopene, cysteine, phosphonate compounds, e.g., etidronic acid, desferoxamine and malate.

### Detailed Description of Embodiments

The present invention discloses a stable pharmaceutical formulation of an antibody comprising buffer, free amino acid(s), sodium chloride and surfactant.

In an embodiment, the invention discloses an α4β7 antibody formulation comprising free amino acid, preferably hydrophobic or basic amino acids or their combinations or combination of both.

In the above said embodiment, the hydrophobic amino acid is glycine or proline and basic amino acid is arginine or lysine. Further, the combination of amino acid (s) is a combination of arginine and proline, or arginine and lysine, or arginine, lysine, and glycine.

In any of the above said embodiments, the buffer mentioned in the formulation is an organic buffer, inorganic buffer and/or combinations thereof.

In the above mentioned embodiment of the invention, the said organic buffer is histidine buffer, succinate buffer or acetate buffer.

In yet another embodiment of the invention, the inorganic buffer mentioned in the formulation is a phosphate buffer.

In an embodiment, the instant invention discloses an α4β7 antibody formulation, which is stabilized by the combination of free amino acid and sodium chloride.

The formulation disclosed in the invention does not require the presence of any sugar/sugar alcohol, PEG or antioxidant(s).

In all of the above mentioned embodiments of the invention, the concentration of the antibody in the formulation is about 50 mg/ml to about 200 mg/ml. Preferably, the concentration of the antibody in the formulation is about 60 mg/ml, or about 70 mg/ml, or about 80 mg/ml, or 20 about 90 mg/ml, or about 100 mg/ml, or about 110 mg/ml, or about 120 mg/ml, or about 130 mg/ml, or about 140 mg/ml, or about 150 mg/ml, or about 160 mg/ml, or about 170 mg/ml or about 180 mg/ml or 190 mg/ml or about 200 mg/ml.

In an embodiment, the invention discloses a high concentration α4β7 antibody formulation comprising about 150 mg/ml to about 180 mg/ml α4β7 antibody, histidine-phosphate buffer, salt, free amino acid(s) and surfactant.

In another embodiment, the invention discloses high concentration, α4β7 antibody formulation comprising about 150 mg/ml to about 180 mg/ml α4β7 antibody, histidine-phosphate buffer, salt, arginine and surfactant.

In an embodiment, the invention discloses a high concentration α4β7 antibody formulation comprising 160 mg/ml to 175 mg/ml α4β7 antibody, histidine-phosphate buffer, salt, arginine, and surfactant.

In yet embodiment, the invention discloses high concentration, α4β7 antibody formulation comprising about 150 mg/ml to about 180 mg/ml α4β7 antibody, histidine-phosphate buffer, salt, arginine, proline and surfactant.

In another embodiment, the invention discloses high concentration, α4β7 antibody formulation comprising about 150 mg/ml to about 180 mg/ml α4β7 antibody, histidine-phosphate buffer, salt, arginine, lysine, glycine and surfactant.

In any of the above said embodiments of the invention, the α4β7 antibody formulation is stable and maintains at least 98% of monomeric content of the antibody, even after storage at 40° C. for two weeks and also change in aggregate content of the antibody is less than 1% even after storage at 40° C. for two weeks.

In any of the above said embodiments of the invention, the α4β7 antibody formulation is stable and maintains at least 97% of monomeric content of the antibody, even after storage at 40° C. for four weeks and also change in aggregate content of the antibody is less than 1.5% even after storage at 40° C. for four weeks.

In any of the above said embodiments of the invention, the α4β7 antibody formulation is stable and contains less than 0.5% of low molecular weight (LMW) species or fragments in the formulation, even after storage at 40° C. for two weeks.

In any of the above said embodiments of the invention, the α4β7 antibody formulation is stable and contains less than 1.2% of low molecular weight (LMW) species or fragments in the formulation, even after storage at 40° C. for four weeks.

In any of the above said embodiments of the invention, the combination of amino acid, salt and surfactant present in the formulations inhibits/reduces the formation of charge variants even after storage at 40° C. for two to four weeks.

In an embodiment, the invention discloses high concentration, α4β7 antibody formulation comprising about 150 mg/ml to about 180 mg/ml α4β7 antibody, histidine-phosphate buffer, salt, arginine, lysine, and surfactant.

In an embodiment, the invention discloses a high concentration α4β7 antibody formulation, comprising about 150 mg/ml to 180 mg/ml of α4β7 antibody, histidine-phosphate buffer, 20 mg/ml-30 mg/ml arginine, 1 mg/ml to 6 mg/ml sodium chloride, and surfactant, wherein the formulation is free of sugar, or PEG, or anti-oxidant. The disclosed combination of excipients in the antibody formulation maintains at least 97% of monomeric content of the antibody composition even after storage at 40° C. for two to four weeks.

In another embodiment, the invention discloses a room temperature stable high concentration α4β7 antibody formulation, comprising about 150 mg/ml to 180 mg/ml of α4β7 antibody, histidine-phosphate buffer, 20 mg/ml-30 mg/ml arginine, 1 mg/ml to 6 mg/ml sodium chloride, and surfactant, wherein the antibody formulation is free of sugar, or PEG or anti-oxidant. The disclosed combination of excipients in the antibody formulation maintains at least 98% of monomeric content of the antibody composition even after storage at 25° C. for four weeks.

In any of the above said embodiments of the invention, the combination of arginine and sodium chloride present in the formulation imparts colloidal stability to the antibody formulation.

In any of the above said embodiments, the viscosity of formulations is less than 20 cP, specifically less than 10 cP.

In an embodiment the invention discloses, a method of controlling conversion of main peak content to charge variants and aggregate formation of an α4β7 antibody in an antibody formulation, wherein the method comprises addition of a buffer composition comprising arginine, sodium chloride and surfactant to the said antibody formulation. The method does not require a sugar/sugar alcohol or PEG or anti-oxidant(s) to control the charge variants or aggregate content of the antibody.

In another embodiment, the invention discloses a method of imparting colloidal stability to α4β7 antibody in an α4β7 antibody formulation by formulating the antibody in a composition comprising arginine, sodium chloride and surfactant.

In yet another embodiment, the invention discloses a method of controlling charge variants of α4β7 antibody in an α4β7 antibody formulation, wherein the method process comprises addition of a buffer composition comprising arginine, sodium chloride and surfactant to the antibody formulation, wherein the formulation formulated by the said method, controls formation of charge variants and at least 50% of the antibody as main peak content when stored at 40° C. for two weeks.

In an embodiment, the invention discloses a method of controlling aggregate formation/content of α4β7 antibody in an α4β7 antibody formulation, wherein the method comprising addition of a buffer composition comprising arginine, sodium chloride and surfactant to the antibody formulation, wherein the formulation formulated by the said method, controls aggregate formation of the antibody and maintains at least 98% of monomeric content of the antibody as main peak content when stored at 40° C. for two weeks.

In the above said embodiment, the α4β7 antibody formulation is stable and maintains at least 98% of monomeric content of the antibody, even after storage at 40° C. for two weeks and also change aggregate content of the antibody is less than 1% even after storage at 40° C. for two weeks.

In another embodiment, the invention discloses a method of controlling charge variants of α4β7 antibody in an α4β7 antibody formulation, wherein the method comprising addition of a buffer composition comprising arginine, sodium chloride and surfactant to the antibody formulation, wherein the formulation formulated by the said method, controls formation of charge variants and maintains at least 45% of the antibody as main peak content when stored at 40° C. for four weeks.

In an embodiment, the invention discloses a method of controlling-aggregate formation/content of α4β7 antibody in an α4β7 antibody formulation, wherein the process comprising addition of a buffer composition comprising arginine, sodium chloride and surfactant to the antibody formulation, wherein the formulation formulated by the said method, controls aggregate formation of the antibody and maintains at least 97% of monomeric content of the antibody as main peak content when stored at 40° C. for four weeks.

In the above said embodiment, the α4β7 antibody formulation is stable and maintains at least 97% of monomeric content of the antibody, even after storage at 40° C. for four weeks and also change in aggregate content of the antibody is less than 1.5% even after storage at 40° C. for four weeks.

In any of the above said embodiments, the α4β7 antibody formulated in combination of sodium chloride, arginine and surfactant is biologically active.

In any of the above mentioned embodiments of the invention, the pH of α4β7 antibody formulation is from pH 5.0-pH 7.0.

In any of the above mentioned embodiments of the invention, pH of the α4β7 antibody formulation is from pH 6.0 to pH 7.0.

In any of the above mentioned embodiments, the formulation of α4β7 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of α4β7 antibody can be reconstituted with appropriate diluent to achieve the liquid formulation suitable for administration.

In any of the above mentioned embodiments, the stable liquid α4β7 antibody are compatible with lyophilization process and the lyophilization process does not impact quality attributes of the antibody.

In any of the above mentioned embodiments, the α4β7 antibody is vedolizumab.

Another aspect of the invention provides a vial, pre-filled syringe or autoinjector device, or any other suitable device comprising any of the subject formulations described herein. In certain embodiments, the aqueous formulation stored in the vial or pre-filled syringe or autoinjector device contains vedolizumab, buffer, amino acid, salt and surfactant.

In any of the above mentioned embodiments, α4β7 antibody formulations are visibly clear without any particles even when stored at 40° C. for four weeks or, at 25° C. for four weeks.

The disclosed formulations of the invention uses lesser amounts of excipients to stabilize the therapeutic antibody. Further, the formulations are devoid of sugar, PEG, and anti-oxidant.

Certain specific aspects and embodiments of the invention are more fully described by reference to the following examples. However, these examples should not be construed as limiting the scope of the invention in any manner.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

An α4β7 antibody, vedolizumab, suitable for storage in the present pharmaceutical composition is produced by standard methods known in the art. For example, vedolizumab is prepared by recombinant expression of immunoglobulin light and heavy chain genes in a mammalian host cell such as Chinese Hamster Ovary cells. Further, the expressed vedolizumab is harvested and the crude harvest is subjected to standard downstream process steps that include purification, filtration and optionally dilution or concentration steps. For example, the crude harvest of vedolizumab may be purified using standard chromatography techniques such as affinity chromatography, ion-exchange chromatography and combinations thereof. The purified vedolizumab solution can additionally be subjected to one or more filtration steps, and the solution obtained is subjected to further formulation studies.

### Example 1: High Concentration Vedolizumab Formulations

Purified high concentration vedolizumab antibody approximately 70-100 mg/ml contains at least 8 mg/ml arginine and/or 75 mg/ml trehalose in histidine-phosphate/histidine buffer back ground was obtained from downstream chromatographic steps. Post which, depending on the requirement of excipients in the final formulation, buffer exchanged with a composition comprising histidine/histidine-phosphate buffer with required amount of following excipients NaCl, and one or more free amino acid(s) such as arginine, lysine, glycine, or proline. Post buffer exchange, the formulations were concentrated upto 150-180 mg/ml. After, polysorbate 80 was spiked in all formulations.

To maintain control, approximately 100 mg/ml of purified vedolizumab in histidine buffer back ground containing 26.3 mg/ml arginine, 100 mg/ml sucrose was obtained from downstream chromatographic steps was buffer exchanged with a composition containing histidine buffer, arginine, and citrate. Post which, the antibody was concentrated upto 170 mg/ml. Polysorbate-80 was added to the final formulation. Approved high concentration vedolizumab formulation contains the above composition. Hence, maintained as control.

Details of all the vedolizumab formulations are mentioned in Table 1. All vedolizumab formulations were subjected for accelerated stability studies at 40° C. for four weeks and at 25° C. for four weeks. Post which, the samples were analyzed for low molecular weight (LMW) species and monomer content using size exclusion chromatography (SEC) [results are given in Table 2 and Table 3] and also checked for main peak content, acidic, basic variants using ion-exchange chromatography [Table 4 and 5].

TABLE 1
Compositions of various high concentration vedolizumab
formulations without sugars and anti-oxidant

| Sample | |
|---|---|
| Name | Composition |
| Vmab- | Vedolizumab 175 mg/ml, 50 mM histidine monohydrochloride, |
| Control | arginine 26.3 mg/ml, 6.7 mg/ml sodium citrate, 0.5 mg/ml |
| | citric acid monohydro chloride, 0.6 mg/mL polysorbate 80, |
| | pH 6.4 |
| Vmab-1 | Vedolizumab 166 mg/ml, 20 mM histidine -phosphate buffer, |
| | 5.82 mg/ml NaCl, 26 mg/ml arginine hydochloride, 0.6 mg/mL |
| | polysorbate 80, pH 6.4 |
| Vmab-2 | Vedolizumab 173.7 mg/ml, 20 mM histidine -phosphate buffer, |
| | 1.46 mg/ml NaCl, 25.2 mg/ml arginine hydrochloride, 5 mg/ml |
| | lysine, 0.6 mg/mL polysorbate 80, pH 6.4 |
| Vmab-3 | Vedolizumab 169.6 mg/ml, 20 mM histidine -phosphate buffer, |
| | 1.46 mg/ml NaCl, 25.2 mg/ml arginine hydrochloride, 3 mg/ml |
| | proline, 0.6 mg/mL polysorbate 80, pH 6.4 |
| Vmab-4 | Vedolizumab 174.8 mg/ml, 20 mM histidine -phosphate buffer, |
| | 1.75 mg/ml NaCl, 25.2 mg/ml arginine hydrochloride, 1 mg/ml |
| | lysine, 1 mg/ml glycine, proline, 0.6 mg/mL polysorbate 80, |
| | pH 6.4 |

TABLE 2
SEC data of high concentration vedolizumab
formulations prepared as per example 1

| | SEC data at 40° C. | | | | | | | | |
|---|---|---|---|---|---|---|---|---|---|
| Sample | % of LMW | | | % of monomer | | | % of HMW | | |
| Name | 0 W | 2 W | 4 W | 0 W | 2 W | 4 W | 0 W | 2 W | 4 W |
| Vmab- | 0.07 | 0.4 | 0.9 | 99.47 | 98.7 | 97.9 | 0.5 | 0.9 | 1.2 |
| Control | | | | | | | | | |
| Vmab-1 | 0.06 | 0.5 | 1.1 | 99.33 | 98.3 | 97.5 | 0.4 | 0.9 | 1.4 |
| Vmab-2 | 0.1 | 0.5 | 0.8 | 99.2 | 97.8 | 96.5 | 0.7 | 1.8 | 2.7 |
| Vmab-3 | 0.0 | 0.4 | 0.8 | 99.3 | 97.9 | 97.0 | 0.7 | 1.6 | 2.2 |
| Vmab-4 | 0.0 | 0.4 | 0.8 | 99.4 | 98.1 | 97.1 | 0.6 | 1.5 | 2.1 |

W—indicates weeks,

TABLE 3
SEC data of high concentration vedolizumab
formulations prepared as per example 1

| | SEC data at 25° C. | | |
|---|---|---|---|
| Sample | % of LMW | % of monomer | % of HMW |

| Name | 0 W | 4 W | 0 W | 4 W | 0 W | 4 W |
|---|---|---|---|---|---|---|
| Vmab- | 0.07 | 0.2 | 99.47 | 99.1 | 0.5 | 0.7 |
| Control | | | | | | |
| Vmab-1 | 0.06 | 0.2 | 99.33 | 99.2 | 0.4 | 0.6 |
| Vmab-2 | 0.1 | 0.2 | 99.2 | 98.7 | 0.7 | 1.1 |
| Vmab-3 | 0.0 | 0.2 | 99.3 | 98.7 | 0.7 | 1.1 |
| Vmab-4 | 0.0 | 0.2 | 99.4 | 98.8 | 0.6 | 1.0 |

W—indicates weeks

TABLE 4
IEX data of high concentration vedolizumab
formulations prepared as per example 1

IEX data at 40° C.

| Sample | % of basic variants | | | % of main peak | | |
|---|---|---|---|---|---|---|
| Name | 0 W | 2 W | 4 W | 0 W | 2 W | 4 W |
| Vmab- | 10.0 | 16.6 | 12.2 | 68.1 | 52.1 | 42.5 |
| Control | | | | | | |
| Vmab-1 | 9.5 | 16.5 | 12.2 | 67.6 | 52.7 | 45.4 |
| Vmab-2 | 11.8 | 13.4 | 15.4 | 68.0 | 54.0 | 44.0 |
| Vmab-3 | 11.4 | 13.1 | 15.2 | 67.3 | 53.8 | 43.7 |
| Vmab-4 | 11.0 | 11.6 | 13.9 | 68.5 | 56.4 | 45.8 |

W—indicates weeks,

TABLE 5
IEX data of high concentration vedolizumab
formulations prepared as per example 1

| IEX data at 25° C. | |
|---|---|
| % of basic variants | % of main peak |

| Sample Name | 0 W | 4 W | 0 W | 4 W |
|---|---|---|---|---|
| Vmab-Control | 10.0 | 10.7 | 68.1 | 64.9 |
| Vmab-1 | 9.5 | 10.2 | 67.6 | 64.5 |
| Vmab-2 | 11.8 | 10.8 | 68.0 | 62.8 |
| Vmab-3 | 11.4 | 11.9 | 67.3 | 61.0 |
| Vma-4 | 11.0 | 9.1 | 68.5 | 64.7 |

W—indicates weeks

All the above formulations were also checked for change in pH. It was observed that there is no change in pH of the formulations even after storage for four weeks at 40° C. and also at 25° C. for four weeks.

Further, all the samples were checked for visible particles. It was observed that, all the samples were clear to slightly opalescent, colorless without any visible particles.

1. A pharmaceutical formulation of an α4β7 antibody comprising, an α4β7 antibody, a buffer having pH of 6.0 to 7.0, free amino acid(s), sodium chloride and surfactant.

2. The formulation as claimed in claim 1, is devoid of sugar, polyethyleneglycol or antioxidant.

3. (canceled)

4. (canceled)

5. The formulation as claimed in claim 1, wherein the α4β7 antibody is vedolizumab.

6. (canceled)

7. The formulation as claimed in claim 1, wherein the α4β7 antibody is at a concentration ranging from 160 mg/ml to 175 mg/ml.

8. The formulation as claimed in claim 1, is stable and exhibits stability under following storage conditions at 40° C. for four weeks or at 25° C. for four weeks.

9. A method of controlling aggregate formation of an α4β7 antibody in a high concentration α4β7 antibody formulation, the method comprises addition of a buffer composition comprising arginine, sodium chloride and surfactant to the antibody formulation, wherein the formulation formulated by the said method controls formation of aggregates of the antibody to less than 2% when stored at 40° C. for four weeks or at 25° C. for four weeks.

10. A method of controlling formation of charge variants of α4β7 antibody in a high concentration α4β7 antibody formulation, the method comprises addition of a buffer composition comprising arginine, sodium chloride and surfactant to the antibody formulation, wherein the formulation formulated by the said method controls formation of charge variants and maintains at least 45% of the antibody as main peak content when stored at 40° C. for four weeks or at 25° C. for four weeks.

11. The formulation as claimed in claim 1, wherein the free amino acid is arginine.

12. The method as claimed in claim 9, wherein the α4β7 antibody is vedolizumab

13. The method as claimed in claim 9, wherein the α4β7 antibody is at a concentration ranging from 160 mg/ml to 175 mg/ml.

14. The method as claimed in claim 10, wherein the α4β7 antibody is vedolizumab

15. The method as claimed in claim 10, wherein the α4β7 antibody is at a concentration ranging from 160 mg/ml to 175 mg/ml.
